(19) United States
(12) Patent Application Publication
(10) Pub. No.: US 2009/0221513 A1
(43) Pub. Date: Sep. 3, 2009

(54) Treatment of Neurodegenerative Disorders

(75) Inventors: Steven Peter Russell Rose, Milton Keynes (GB); Radmila Mileusnic, Buckinghamshire (GB)

Correspondence Address: WOLF GREENFIELD & SACKS, P.C., 600 ATLANTIC AVENUE, BOSTON, MA, 02210-2206, US

(73) Assignee: The Open University, Milton Keynes (GB)

(21) Appl. No.: 12/085,480

(22) PCT Filed: Nov. 24, 2006
(86) PCT No.: PCT/GB2006/050414

§ 371 Date: May 8, 2009

(30) Foreign Application Priority Data
Nov. 25, 2005 (GB) 0524023.9

(51) Int. Cl. A61K 38/06; A61P 25/00

(52) U.S. Cl. 514/18

(57) ABSTRACT

Peptides having the sequence D-Arg-L-Glu-L-Arg, or the sequence L-Arg-D-Glu-L-Arg and derivatives thereof, are disclosed. Such peptides are useful in treatment of neurodegenerative disorders, and as cognitive enhancers. Preferred peptides include a protective group.

### FIELD OF THE INVENTION

The present invention relates to a compound comprising a tripeptide, and derivatives thereof, and to the use of such a compound in treatment of neurodegenerative disorders.

### BACKGROUND TO THE INVENTION

Alzheimer's disease is a degenerative brain disease which is characterised by progressive loss of memory and subsequently most other cognitive functions in an irreversible decline over a period of years. It represents a substantial health problem, particularly in an ageing population and currently affects some 800,000 people in the UK alone.

Until recently, therapeutic approaches for Alzheimer's disease have addressed the stabilisation of acetylcholine concentration. The use of acetylcholine esterase inhibitors results in a temporary improvement which is not suitable for stopping or reversing the degeneration. The efficacy of these drugs has been criticised by NICE (the UK's National Institute for Clinical Excellence) and there is urgent need for more effective approaches, based on greater understanding of the biochemical mechanisms underlying the neuronal cell death that characterises the disease.

Two effects which have been noted to take place in the brain of a person suffering from Alzheimer's disease are the build up outside the nerve cells of the brain of tangled masses of protein (plaques) and the build up inside the brain cells of a different protein (neurofibrillary tangles). The extracellular proteins are known to be aggregates of polypeptides having amino acid sequences corresponding to the amyloid-beta portions of the amyloid precursor protein APP. The tangled masses of these proteins are known as amyloid plaques. The intracellular proteins are known as neurofibrillary and tau proteins. It is however not known whether either or both of the extracellular accumulation of amyloid plaques and the intracellular accumulation of neurofibrillary proteins are the causes or the symptoms of Alzheimer's and related neurodegenerative diseases of the Alzheimer type.

The APP family consists of 8 isoforms made of 770, 752, 751, 733, 714, 696, 695 and 677 amino acid residues generated by alternative splicing (see Selkoe, Annu Rev Neurosci 17, 489-517, 1994). The isoform present in neurons is known to consist of 695 amino acid residues in a known sequence [(see Kang et al, Nature 325, 733-736 (1987), and Carrodeguas et al, Neuroscience 134, 1285-1300 (2005), the contents of which are incorporated herein by reference]. Chick and human APP-751 sequences are compared in FIG. 1 of Carrodeguas.

APP is a multifunctional transmembrane protein and is known to have important functions in normal brain tissue including neuritic outgrowth. Downregulating APP synthesis or blocking its extracellular N terminal domain with antibodies prevents long term memory formation in a well established animal model system for the study of the molecular processes involved in memory formation, the one trial passive avoidance task in the young chick (see Mileusnic et al 2000).

The chick form of APP is known to consist of the same number of amino acid residues as and to resemble the human form closely, being approximately 95% homologous therewith. The amino acid sequence from amino acid 360 to 460 of APP is identical in the human and chick forms of APP (see Kang et al 1997, Carrodeguas et al 2005, and Barnes et al, J Neurosci, 18 (15) 5869-5880 (1998), the contents of which are also incorporated herein by reference).

International Patent Application WO02/083729, the contents of which are incorporated herein by reference, reports that amnesia induced in chicks by blocking APP synthesis or function, or by injection of amyloid-beta, can be prevented by injection of a small peptide homologous to part of the growth promoting domain of APP (amino acid residues 375 to 392). It is reported that a particularly preferred peptide is Arg-Glu-Arg (hereafter RER), homologous to residues 328 to 330 of the human APP sequence given in WO02/083729.

The present invention is based on the identification of a further preferred peptide.

### SUMMARY OF THE INVENTION

Amino acids can exist in the naturally occurring L-form (designated in the single letter amino acid code by the use of upper case) or their optical isomeric D-form (designated by the use of lower case). The present inventors have determined that peptides of the sequence rER (that is, D-Arg-L-Glu-L-Arg), and derivatives, are particularly biologically active. In addition, peptides of the sequence ReR (L-Arg-D-Glu-L-Arg) also appear to be biologically active, although to a lesser degree than the peptide rER.

According to a first aspect of the present invention, there is provided a compound comprising a peptide having the sequence rER, or a peptide having the sequence ReR. A particularly preferred peptide has the sequence rER. The peptide may comprise one or more protective groups, preferably an N-terminal protective group. In a preferred embodiment, the protective group is an acyl group, preferably an acetyl group (Ac-rER). Other acyl protective groups may have the formula R—C—, where R represents a straight or branched chain alkyl group, for example a methyl, ethyl, n-propyl, isopropyl, n-butyl, isobutyl, s-butyl, t-butyl, pentyl or hexyl group, a substituted or unsubstituted cycloalkyl group, for example a methylcyclohexyl or cyclohexyl group, a substituted or unsubstituted straight- or branched-chain aralkyl group, for example a benzyl group, or a substituted or unsubstituted aryl group, for example a phenyl or tolyl group. Examples of substituents in the substituted groups mentioned above are the alkyl groups also mentioned above. Other suitable protective groups may be used (for example, Fmoc, Boc, Alloc).

Ac-rER has the structural formula

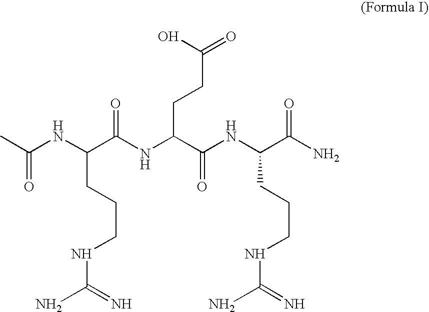

In other embodiments the protective group may be a C-terminal protective group.

Preferably the compound consists essentially of a tripeptide having the sequence rER, optionally with a protective group. Alternatively the compound may consist essentially of a tripeptide having the sequence ReR. However, in certain embodiments the peptide may include additional amino acid residues. The peptide preferably comprises no more than 1, 2, 5, 10, 15, 20, 25, or 30 additional amino acids. In preferred embodiments, the peptide may include residues adjacent the RER sequence found at amino acid residues 328-330 of human APP as described in WO02/083729. Particular preferred additional residues are as described in WO02/083729. For example, the peptide may consist of or comprise any of the sequences rER, rERM, and rERMS; or any of the sequences ReR, ReRM, and ReRMS. Alternatively, or in addition, the peptide may include additional non-standard amino acids, for example, other D-amino acids, or amino acids not naturally occurring in mammals. The peptide may comprise a tripeptide having the sequence rER (or the sequence ReR), optionally with a protective group, conjugated to another peptide sequence unrelated to human APP; for example, an immunoglobulin sequence, a targeting sequence, or the like. The compound may comprise a tripeptide having the sequence rER (or the sequence ReR) conjugated to a non-peptide molecule; for example, a fluorescent, radioactive, or other label.

The invention also provides compounds comprising a derivative of the peptide rER, or a derivative of the peptide ReR. Derivatives may include salts; modified amino acids, in particular amino acids modified by methylation, amidation, acetylation, or substitution with other chemical groups. Preferably the modifications are selected to alter the peptide's circulating half-life without adversely affecting activity. Derivatives may include peptide mimetics, for example where the peptide backbone has been substituted or replaced. In certain embodiments, the peptide backbone may be modified to include a methyl group, giving for example the peptide Ac-rE-(Me)R. Other modifications may be used to enhance stability of the peptide or derivatives. Peptides or derivatives of the present invention may be labelled; for example, by conjugation to a detectable label. Suitable labels include gold or fluorescent markers, markers having enzymatic activity, and the like.

The present invention further provides a compound comprising a peptide having the sequence rER, or the sequence ReR, or a derivative thereof, for use as a medicament. Also provided is the use of a compound comprising a peptide having the sequence rER, or the sequence ReR, or a derivative thereof, in the preparation of a medicament for treatment of a neurodegenerative disorder. Preferably the disorder is Alzheimer's disease. The invention further provides the use of a compound comprising a peptide having the sequence rER, or the sequence ReR, or a derivative thereof, in the preparation of a medicament for enhancing cognitive function.

Further aspects of the present invention relate to methods for treatment of a neurodegenerative disorder, preferably Alzheimer's disease; or to methods for enhancing cognitive function. The methods comprise administering a compound comprising a peptide having the sequence rER, or the sequence ReR, or a derivative thereof, to a subject. Preferably the subject is human. The peptide may be administered in any convenient manner; for example, by subcutaneous injection, intravenous administration, orally, transdermally, nasally, rectally, parenterally, or by pulmonary administration. Suitable dosage levels will depend among other factors on the nature and severity of the disorder to be treated; age, weight, and sex of the subject; the route of administration; and potential interactions with any other treatments the subject is taking. Preferred dosage levels may be from 0.1 to 100 mg active substance per kg of subject bodyweight; preferably from 0.5 to 50 mg/kg; and more preferably from 1 to 25 mg/kg.

According to a further aspect of the invention, there is provided a pharmaceutical formulation comprising a compound comprising a peptide having the sequence rER, or the sequence ReR, or a derivative thereof. The formulation may comprise a pharmaceutically acceptable carrier. Delivery systems which may be used with the invention include, for example, aqueous and non aqueous gels, creams, multiple emulsions, microemulsions, liposomes, ointments, aqueous and non aqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

A pharmaceutical formulation of the invention is in a form suitable for administration, e.g., systemic, topical or local administration, into a cell or subject, including for example a human. Suitable forms, in part, depend upon the use or the route of entry, for example oral, transdermal, or by injection. Other factors are known in the art, and include considerations such as toxicity and forms that prevent the composition or formulation from exerting its effect.

The present invention also includes compositions prepared for storage or administration that include the desired peptide or derivative in a pharmaceutically acceptable carrier or diluent. Acceptable carriers or diluents for therapeutic use are well known in the pharmaceutical art. For example, preservatives, stabilizers, dyes and flavouring agents can be provided. These include sodium benzoate, sorbic acid and esters of p-hydroxybenzoic acid. In addition, antioxidants and suspending agents can be used.

The formulations of the invention can be administered in dosage unit formulations containing conventional non-toxic pharmaceutically acceptable carriers, adjuvants and/or vehicles. Formulations can be in a form suitable for oral use, for example, as tablets, troches, lozenges, aqueous or oily suspensions, dispersible powders or granules, emulsion, hard or soft capsules, or syrups or elixirs. Compositions intended for oral use can be prepared according to any method known to the art for the manufacture of pharmaceutical compositions and such compositions can contain one or more such sweetening agents, flavouring agents, colouring agents or preservative agents in order to provide pharmaceutically elegant and palatable preparations. Tablets contain the active ingredient in admixture with non-toxic pharmaceutically acceptable excipients that are suitable for the manufacture of tablets.

These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more colouring agents, one or more flavouring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavouring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavouring and colouring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavouring agents.

Syrups and elixirs can be formulated with sweetening agents, for example glycerol, propylene glycol, sorbitol, glucose or sucrose. Such formulations can also contain a demulcent, a preservative and flavouring and colouring agents. The pharmaceutical compositions can be in the form of a sterile injectable aqueous or oleaginous suspension.

This suspension can be formulated according to the known art using those suitable dispersing or wetting agents and suspending agents that have been mentioned above.

A sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The compounds of the invention can also be administered in the form of suppositories, e.g. for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The peptides and derivatives of the present invention can also be administered to a subject in combination with other therapeutic compounds to increase the overall therapeutic effect. The use of multiple compounds to treat an indication can increase the beneficial effects while reducing the presence of side effects.

According to a still further aspect of the present invention, there is provided an antibody which specifically binds to a peptide having the sequence rER, or an antibody which specifically binds to a peptide having the sequence ReR. By ‘specifically binds’ is meant that the antibody binds to the peptide at a level that is significantly greater than any non-specific binding that may be observed. It will be understood by those of skill in the art that an antibody specific for the rER peptide (or the ReR peptide) may nonetheless still bind other antigens having a similar epitope. Specific antibodies may be prepared by immunising a subject mammal with a preparation comprising the rER peptide (or the ReR peptide) of the invention. The antibody of the invention may be polyclonal or monoclonal. The antibodies of the invention may comprise recombinant, chimeric, or humanised antibodies. The invention also provides immunologically active fragments of such antibodies; in particular F(ab) and F(ab′)₂ fragments.

Antibodies of the invention may be used to screen other compounds, in order to identify potential candidate molecules having similar activity to the rER peptide. Thus, the invention also provides a method for identifying a compound having activity useful for the treatment of neurodegenerative disorders, or useful in the enhancement of cognitive function, the method comprising contacting a candidate compound with an antibody specific for a peptide having the sequence rER, or the sequence ReR, and determining whether the antibody binds the compound.

### BRIEF DESCRIPTION OF THE DRAWINGS

These and other aspects of the present invention will now be described by way of example only and with reference to the accompanying drawings, which show:

FIG. 1 The effect of different D/L forms of tripeptide as memory enhancers on weak memory

FIG. 2 The effect of Ac-rER on memory in chicks with Aβ-induced amnesia.

FIG. 3 The effect of Ac-rER as memory enhancer in chicks trained on a weak training task (WT).

FIG. 4 Distribution of Fluorescein labelled Ac-rER in chick brain. The tripeptide was injected ip and ic.

FIG. 5 Dose-dependence of the effect of Ac-rER in weak training

FIG. 6 Stability of Ac-rER

FIG. 7 Effect of Ac-rER on Anisomycin-induced amnesia in chicks

FIG. 8 Effect of Ac-rER on MK801-induced amnesia

FIG. 9 Effect of ic injected Ac-rE(Me)R on weak training (WT).

FIG. 10 Effect of ip injected Ac-rE(Me)R on weak training (WT).

### DETAILED DESCRIPTION OF THE DRAWINGS

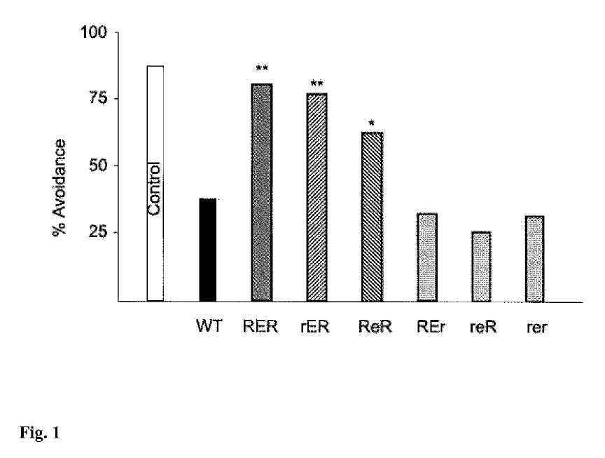

FIG. 1 The effect of different D/L forms of tripeptide as memory enhancers on weak memory. Chicks were injected ic with different D/L forms of tripeptide 60 min pre-training and tested 24 after. The control group received saline. Retention was calculated as the percent in each group which showed avoidance and discrimination. Each chick was trained and tested only once and differences between groups were tested for statistical significance by G-test (Sokal, and Rohlf, 1995). Note that the G-test reflects group differences, and so there are no error bars on the figures.

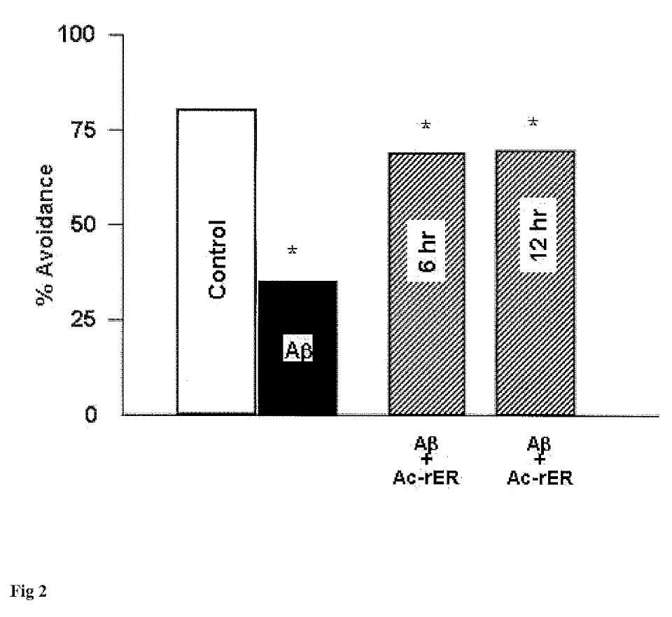

FIG. 2 The effect of Ac-rER on memory in chicks with Aβ-induced amnesia. Chicks were injected 2×ip, 1 mg/100 gr bw, with Ac-rER, 6 hr and 12 hr pre-training. Aβ1-42 (2 μg/hemisphere) was injected ic 60 min pre-training. Chicks were tested 24 hr later. Retention was calculated as the percent in each group which showed avoidance and discrimination. Each chick was trained and tested only once and differences between groups were tested for statistical significance by G-test (Sokal, and Rohlf, 1995).

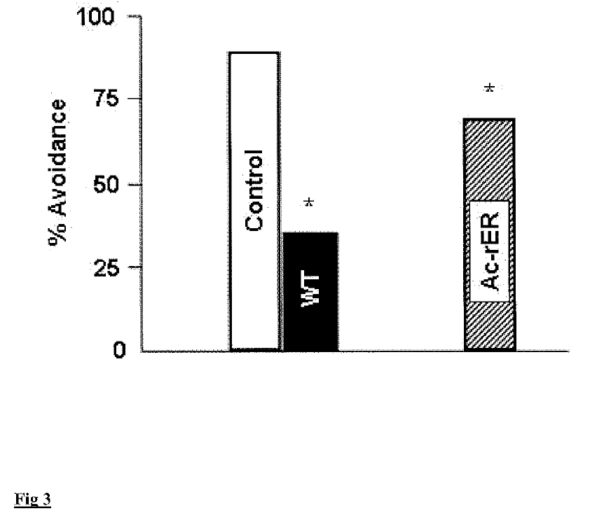

FIG. 3 The effect of Ac-rER as memory enhancer in chicks trained on a weak training task (WT). Chicks were injected 2×ip injections, 1 mg/100 gr bw, 30 min and 6 hr pre-training and tested 24 hr later. The control groups received Ac-REr. Retention was calculated as the percent in each group which showed avoidance and discrimination. Each chick was trained and tested only once and differences between groups were tested for statistical significance by G-test (Sokal, and Rohlf, 1995).

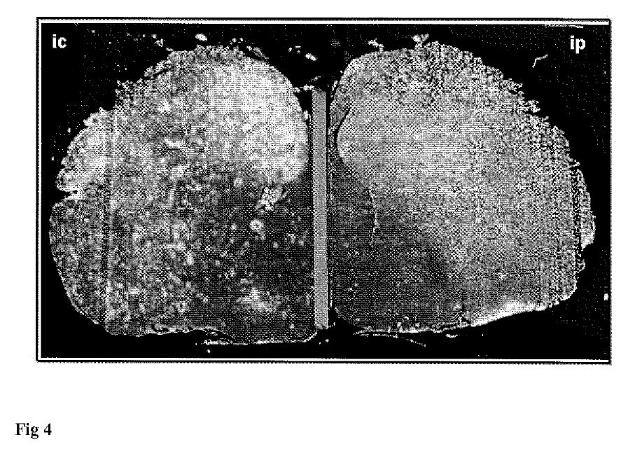

FIG. 4 Distribution of Fluorescein labelled Ac-rER in chick brain. The Fluorescein-Ahx-Ahx-rER was injected ip (2 mg/100 gr bw) and ic (8 μg/hemisphere) 6 hr before sectioning the brains analysis of the distribution of the Fluorescein-labelled rER. The left panel shows the distribution of the Fluorescein-labeled rER after ic injection. The panel on the right shows the distribution of the Fluorescein-labelled rER after ip injection. Note that the distribution of the fluorescence is almost identical.

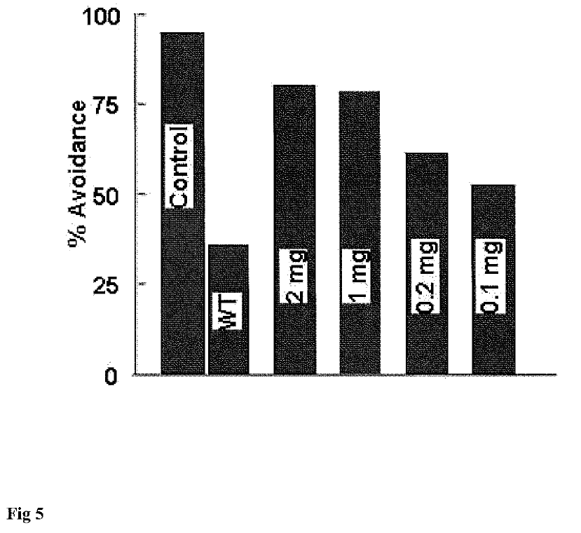

FIG. 5 Dose-dependence of the effect of Ac-rER in weak training. Chicks were injected ip with different doses of the Ac-rER 60 min pre-training and tested 24 after. The control group received saline. Retention was calculated as the percent in each group which showed avoidance and discrimination. Each chick was trained and tested only once and differences between groups were tested for statistical significance by G-test (Sokal, and Rohlf, 1995).

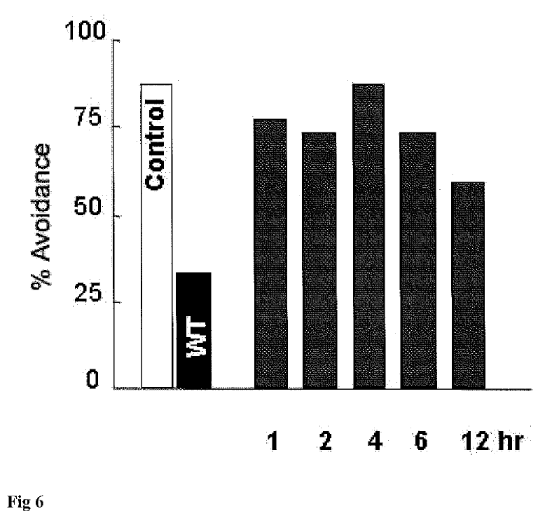

FIG. 6 Stability of Ac-rER. Chicks were injected ip with 2 mg/100 g bw of Ac-rER 1, 2, 4, 6 and 12 hr before training and tested 24 hr later. The control group received saline. Retention was calculated as the percent in each group which showed avoidance and discrimination. Each chick was trained and tested only once and differences between groups were tested for statistical significance by G-test (Sokal, and Rohlf, 1995).

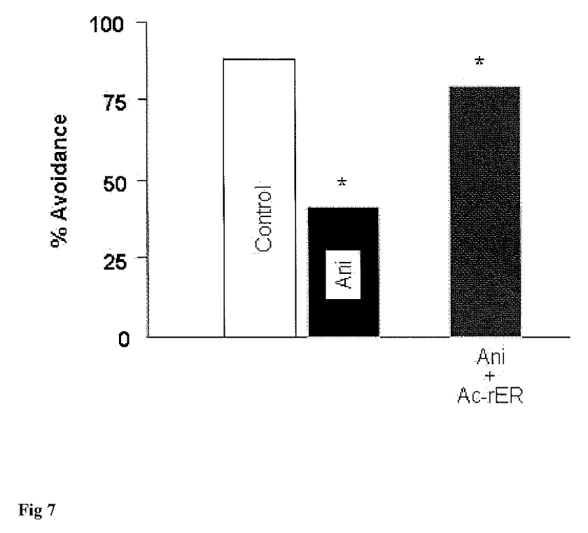

FIG. 7 Effect of Ac-rER on Anisomycin-induced amnesia in chicks. Chicks were injected ic with 8 μg/hemisphere of Ac-rER 60 min pre-training followed by ic injection of Anisomycin (125 nmol/hemisphere). immediately post-training. Controls were injected with saline. Chicks were tested 3 hr post training. Retention was calculated as the percent in each group which showed avoidance and discrimination. Each chick was trained and tested only once and differences between groups were tested for statistical significance by G-test (Sokal, and Rohlf, 1995).

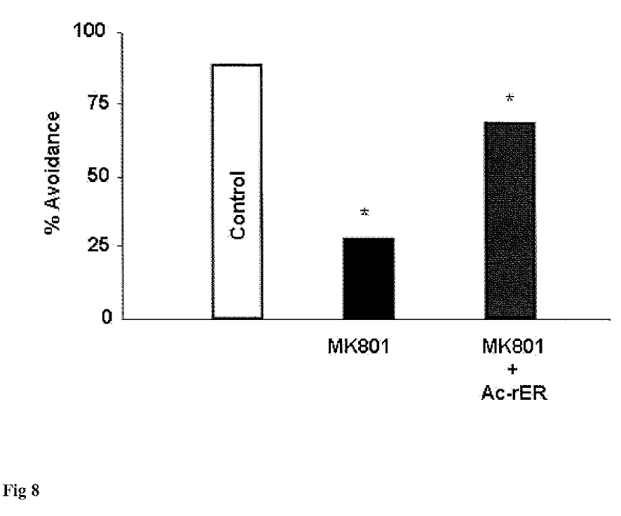

FIG. 8 Effect of Ac-rER on MK801-induced amnesia. Chicks were injected ic with 8 μg/hemisphere of Ac-rER 60 min pre-training followed by ip injection of MK80 1 (0.020 mg/100 gr) pre-training. Controls were injected with saline. Chicks were tested 3 hr later. Retention was calculated as the percent in each group which showed avoidance and discrimination. Each chick was trained and tested only once and differences between groups were tested for statistical significance by G-test (Sokal, and Rohlf, 1995).

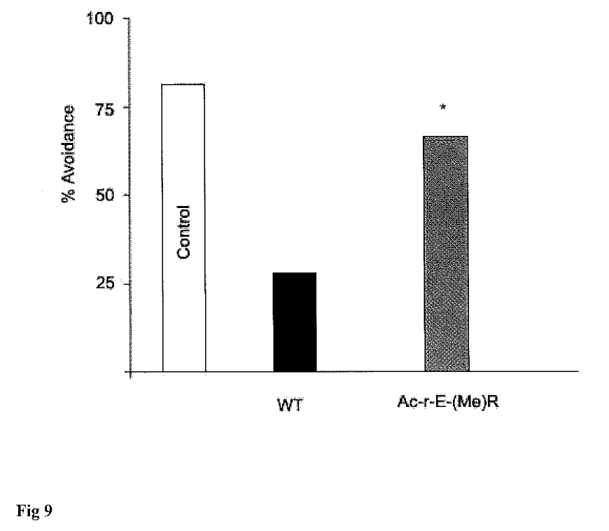

FIG. 9 Effect of ic injected Ac-rE(Me)R on weak training (WT). Chicks were injected ic with 8 μg/hemisphere of Ac-rE(Me)R 60 min pre-training and tested 24 hr later. Controls were injected with saline. Retention was calculated as the percent in each group which showed avoidance and discrimination. Each chick was trained and tested only once and differences between groups were tested for statistical significance by G-test (Sokal, and Rohlf, 1995).

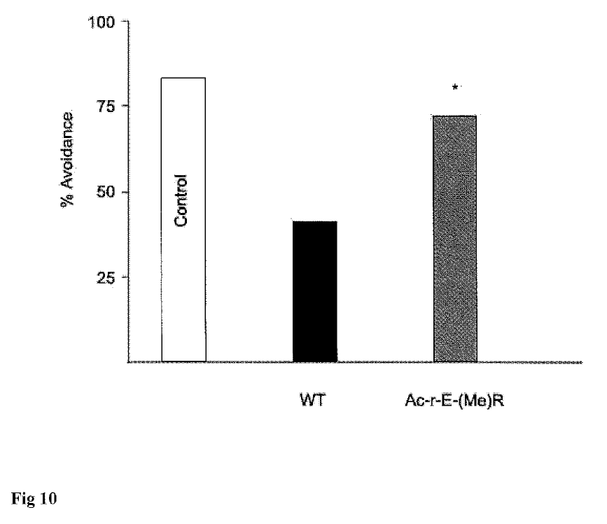

FIG. 10 Effect of ip injected Ac-rE(Me)R on weak training (WT). Chicks were injected ip with 2 mg/100 gr bw of Ac-rE(Me)R 6 hr pre-training and tested 24 hi later. Controls were injected with saline. Retention was calculated as the percent in each group which showed avoidance and discrimination. Each chick was trained and tested only once and differences between groups were tested for statistical significance by G-test (Sokal, and Rohlf, 1995).

### Materials and Methods

### Animals and Training

Commercially obtained Ross Chunky eggs were incubated and hatched in brooders and held until 16±6 hours old. Chicks were placed in pairs in small aluminium pens. Following an equilibration period of an hour, the chicks were pretrained and trained essentially as described by Lossner and Rose (J. Neurochem. 41, 1357-1363 (1983), the contents of which are incorporated herein by reference). Pretraining involved three 10 s presentations of a small (2 mm diameter) white bead, at approximately 5 minute intervals. Chicks which failed to peck the bead at least twice in three presentations (less than 5%), were not used subsequently, but remained in their pens for the duration of the experiment. Two training techniques were used: “strong” and “weak” training. In both, 5 to 10 minutes after the last pre-training trial, chicks were trained by a 10 s presentation of a 4 mm diameter chrome bead, which had been dipped in the bitter-tasting methylanthranilate. Control chicks pecked at a water-coated or dry bead. In the “strong” version of the task, 100% methylanthranilate was used. In the “weak” version, 10% methylanthranilate was used. Chicks spontaneously pecked at the training or control beads within 20 s. Chicks that pecked at the bitter bead evinced a disgust reaction and would not normally peck at a similar, but dry bead for some hours subsequently. At various times following training chicks were tested, by offering them a dry 4 mm diameter chrome bead, followed 10 minutes later by a small (2 mm diameter) white bead, each for 20 to 30 s. Animals were tested by an experimenter blind as to which treatment each chick had received. Chicks are considered to remember the task if they avoid the chrome bead at test but peck at the white bead (discriminate), and to have forgotten it if they peck at both beads. Recall is calculated as a percent avoidance score (percentage of chicks which avoid the chrome bead) and as a discrimination score (percentage of chicks which avoid the chrome but peck at the white bead). The use of the discrimination score ensures that chicks can indeed see and peck accurately at the bead; and hence that the avoidance of the chrome bead is not due to non-specific factors such as lack of visuo-motor coordination, motivation, attention, arousal, etc. but is a positive act, demonstrating memory for the distasteful stimulus. Each chick was trained and tested only once and differences between groups tested for statistical significance by g-test described by Sokal and Rohlf (biometry: the Principles and Practice of Statistics in Biological Research (2nd edition), W H Freeman, New York (1981)), the contents of which are incorporated herein by reference. The validity of this particular training task used to assess memory formation is extensively discussed by Andrew (Neural and Behavioural Plasticity: the Use of the Domestic Chick as a Model, Oxford University Press, Oxford, UK (1991)), the contents of which are incorporated herein by reference.

Chicks trained on the strong version of the task were found to recall the avoidance for at least 48 hours, and more than 80% were found normally to avoid and discriminate on test at 24 hours. Therefore if agents that are amnesic—that is, cause the chick not to remember—are administered, chicks will demonstrate forgetting by pecking rather than avoiding the chrome bead on test. By contrast, chicks were found normally to remember the “weak” version of the task for only a few hours—some 6 to 8 hours in all; retention at 24 hours was normally reduced to some 20 to 30%. Thus the learning experience is not committed to long-term memory. Agents that are memory enhancers can thus be tested. A memory enhancing agent, administered to a chick trained on the weak learning task, produces an increase in retention—increased avoidance of the chrome bead—at 24 hours. That is, such memory enhancers help convert weak to strong reaming by enabling the transition from shorter to longer-term memory.

### Peptide Injections

1. Intracranial (ic) injections: Bilateral intracranial injections (8 μg in 2 μl/hemisphere) of APP-derived peptides were injected intracerebrally into a specific brain region known to be required for memory formation (the intermediate hyperstriatum ventrale) at different time-points pre- or post-training using a 5 μg Hamilton syringe fitted with a plastic sleeve to allow a penetration of 3 mm. After completion of the injection, the needle was kept in place for 5 s. Correct placement was ensured by using a specially designed head holder described by Davis et al (Physiol. Behav. 22, 177-184 (1979), the contents of which are incorporated herein by reference) and was routinely visually monitored post-mortem.
2. Peripheral (ip) injections: Test peptides or other substances were administered intraperitoneally (0.2 ml/chick) using a 1 ml hypodermic syringe at various times either before or after the training protocol. After the completion of injection, the needle was kept in place for 3 sec. Chicks were tested at different time points post-training as described above. The general behaviour of the chicks following injections was observed to detect any potential non-specific or adverse reactions to the injections.

### Peptide Materials

The polypeptides administered were synthesised using a conventional peptide synthesiser in a manner which is well-known to those skilled in the art. The synthesised polypeptides were purified by use of RP-HPLC and purity further checked by mass spectrometry (MALDI-TOF), both techniques being well known to those skilled in the art. The polypeptides after synthesis were kept under argon in a lyophilised state, the argon preventing oxidation of cysteine, methionine and tryptophan in particular.

### Experimental Results

International Patent Application WO02/083729 describes a number of peptides derived from APP. The small peptide RER in particular was shown to be effective as a cognitive enhancer and protector against amyloid beta induced memory loss. We wished to investigate more stable forms of the peptide which could better serve as potential therapeutic agents.

The standard approach to stabilise peptides is to N-terminally protect the molecule. This was achieved by acylation. Ac-RER was effective as a cognitive enhancer and in protecting against amyloid-beta.

We then decided to investigate the bio-activity of the d-isomeric form of the peptide. D-isomers are often toxic and do not normally show similar bio-activity as the naturally occurring L-forms. We synthesised the following D/L-forms: D-R-L-E-L-R (rER), L-R-D-E-L-R(ReR), L-R-L-E-D-R (REr) and D-R-D-E-D-R (rer) Only one, rER, and its acylated form Ac-rER, showed bio-activity to a similar degree as RER, being active as a cognitive enhancer and protecting against amyloid-beta induced memory loss. The ReR form of the peptide showed lesser bio-activity, but still provided an improved effect when compared with no peptide, or with the other forms of the peptide.

FIG. 1 shows the effect of different D/L forms of the tripeptides on memory retention in chicks trained on the weak aversive learning task. Chicks were injected ic with different D/L forms of tripeptide 60 min pre-training and tested 24 after. The control group received saline. The data shows that Ac-D/L/L (Ac-rER) enhances memory retention to the levels observed in the chick treated with the Ac-L/L/L/ form (Ac-RER), while Ac-L/D/L (Ac-ReR) showed significant but lesser effects. All other forms are less biologically active.

FIG. 2 shows the effect of Ac-rER on memory in chicks with Aβ-induced amnesia. Chicks were injected with Ac-rER 2×ip injections, 1 mg/100 gr bw, 6 hr and 12 hr pre-training. Aβ1-42 was injected ic 60 min pre-training. Aβ1-42 is the domain of APP which forms β amyloid plaques, and is described in more detail in Carrodeguas et al (2005). The data shows that Ac-rER injected either 6 or 12 hours pre-training restores cognitive function and prevents memory loss which would otherwise be a result of the Aβ injections. This confirms that Ac-rER protects against the memory loss induced by Aβ, and so may be of benefit in treatment of the cognitive deficits occurring during aging and in neurodegenerative disorders including Alzheimer's disease.

FIG. 3 shows the effect of Ac-rER as memory enhancer in chicks trained on a weak training task (WT). Chicks were injected with 2×ip injections, 1 mg/100 gr bw, 30 min and 6 hr pre-training. The control group received Ac-REr. The results show that performance on the weak training task is significantly enhanced in those animals given Ac-rER.

Importantly from the point of view of potential therapeutic use, these results show that Ac-rER is effective when injected peripherally. To prove that this was because of the ability of Ac-rER to cross the blood-brain barrier and bind to similar sites as does RER, we injected fluorescein labelled Ac-rER ip and ic and six hours later cut sections for fluorescence analysis. FIG. 4 compares the binding of Ac-rER following these two routes of injection. Ac-rER is more stable than Ac-RER as it can be injected up to 12 hr prior to training, and act as cognitive enhancer and neuroprotective agent (FIGS. 2 and 3) as opposed to the 3 hr maximum of the unprotected L-form (as described in WO02/083729).

FIG. 5 shows the effect of different doses of Ac-rER in chicks trained on a weak aversive task. The data shows that a dose as low as 1 mg per 100 g body weight is sufficient to enhance memory in chicks trained on a weak training task.

FIG. 6 shows the persistence of the effect of Ac-rER when administered to chicks trained on a weak training task. The peptide is effective when a single injection of 1-2 mg/100 g bw is given as much as 12 hours before training. The data also suggests that the peptide is most effective when administered between 2 and 6 hours, and preferably 4 hours, before training.

We have also explored the effectiveness of Ac-rER in reversing or protecting against amnesia induced by general protein synthesis inhibitors (anisomycin; FIG. 7) and blockers of NMDA receptors (MK801; FIG. 8), both well-known amnestic agents. To our knowledge there is no known agent available to reverse the amnestic effects of these substances. Anisomycin was administered ic immediately post training, MK801 was given ip 20 min pre-training while the Ac-rER was administered ic 30 min pre-training in experiments described in FIGS. 7 and 8. The results demonstrate that Ac-rER prevents either of these drugs from inducing amnesia.

Finally, we have also explored the effectiveness of Ac-rE(Me)R in weak training(FIGS. 9 and 10). Replacement of the hydrogen bond within the peptide backbone with the N-methyl group should enhance even further the stability of the D/L tripeptide. Ac-r-E-(Me)R was administered ic or ip 6 hr pre-training as described in FIG. 4 and chicks were tested 24 hr later. FIGS. 9 (ic administration) and 10 (ip administration) compare the effect of Ac-rER following two routes of injection and show that the methylated analog of the Ac-rER, Ac-rE(Me)R is tolerated even if there is no hydrogen bond in the Ac-r-E-(Me)R and that this molecule enhances memory in both experimental conditions.

### CONCLUSION

These results demonstrate that Ac-rER and Ac-ReR provide a beneficial effect to enhance cognition and learning in normal animals, as well as preventing induced amnesia. The action of Ac-rER against AD induced amnesia suggests that the peptide will be effective in reducing the cognitive deficits associated with aging and neurodegeneration, and so as a therapeutic agent for treatment of Alzheimer's disease. There is no way of predicting the surprising enhanced effect of the rER peptide in advance from the known activity of the original RER peptide, and discovery of the forms of the peptide that are active compared with the inactive ones provides valuable information about the steric configurations of the molecule that are required to bind to its putative receptor sites on the neuronal membrane.

It will be understood that the foregoing is for illustrative purposes only, and that various modifications may be made to the agents disclosed herein without departing from the scope of the invention. In particular, longer peptides and peptidomimetics incorporating the rER or ReR sequence may be used. Additional modifications may be made to the rER or ReR peptide, for example to enhance stability or bioavailability. One such modification is the incorporation of methyl groups on the peptide backbone. The peptides may incorporate additional amino acid residues, preferably from the human APP protein, and preferably also from the region of the APP protein adjacent the RER motif. Alternatively, or in addition, amino acid residues from other proteins may be incorporated, as may non-peptide molecules.

1. A method for enhancing cognitive function in a normal subject, comprising administering a compound comprising a peptide having the sequence rER (D-Arg-L-Glu-L-Arg), or the sequence ReR (L-Arg-D-Glu-Arg) to a subject.

2. A method for enhancing memory in a normal subject, comprising administering a compound comprising a peptide having the sequence rER (D-Arg-L-Glu-L-Arg), or the sequence ReR (L-Arg-D-Glu-Arg) to a subject.

3. A method for the treatment of impaired memory in a subject, comprising administering a compound comprising a peptide having the sequence rER (D-Arg-L-Glu-L-Arg), or the sequence ReR (L-Arg-D-Glu-Arg) to a subject.

4. The method of claim 3, wherein the impaired memory is amnesia.

5. A method for identifying a compound having activity useful for the treatment of impaired memory or useful in the enhancement of cognitive function, the method comprising contacting a candidate compound with an antibody specific for a peptide having the sequence rER (D-Arg-L-Glu-L-Arg), or with an antibody specific for a peptide having the sequence ReR (L-Arg-D-Glu-Arg), and determining whether the antibody binds the compound.

6. The method of claim 1, wherein the peptide has the sequence rER.

7. The method of claim 1, wherein the peptide comprises one or more protective groups.

8. The method of claim 7, wherein the protective group is an N-terminal protective group.

9. The method of claim 7, wherein the protective group is an acyl group.

10. The method of claim 9, wherein the protective group is an acetyl group.

11. The method of claim 1, wherein the peptide is Ac-rER.

12. The method of claim 1, wherein the peptide has the structural formula:

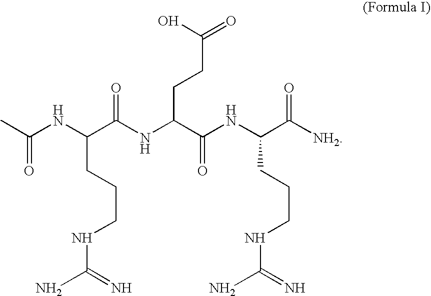

13. The method of claim 1, wherein the peptide consists essentially of a tripeptide having the sequence rER, optionally with a protective group.

14. The method of claim 1, wherein the peptide comprises additional amino acid residues.

15. The method of claim 14, wherein the peptide includes residues adjacent the RER sequence found at amino acid residues 328-330 of human APP.

16. The method of claim 14, wherein the peptide consists of or comprises any of the sequences rER, rERM (SEQ ID NO: 1), and rERMS (SEQ ID NO: 2).

17. The method of claim 14, wherein the peptide comprises additional non-standard amino acids.

18. The method of claim 14, wherein the peptide comprises a tripeptide having the sequence rER, optionally with a protective group, conjugated to another peptide sequence unrelated to human APP.

19. The method of claim 1, wherein the peptide is conjugated to a non-peptide molecule.

20. The method of claim 1, wherein the peptide has a peptide backbone that has been substituted or replaced.

21. The method of claim 20, wherein the peptide backbone includes a methyl group.


SEQUENCE LISTING (4 sequences)

SEQ ID NO: 1 - protein, 4 aa, Homo sapiens
  MISC_FEATURE (1)..(1): Amino acid in the D-form
RERM

SEQ ID NO: 2 - protein, 5 aa, Homo sapiens
  MISC_FEATURE (1)..(1): Amino acid in the D-form
RERMS

SEQ ID NO: 3 - protein, 4 aa, Homo sapiens
  MISC_FEATURE (2)..(2): Amino acid in the D-form
RERM

SEQ ID NO: 4 - protein, 5 aa, Homo sapiens
  MISC_FEATURE (2)..(2): Amino acid in the D-form
RERMS